(19) United States
(12) Patent Application Publication
(10) Pub. No.: US 2023/0301811 A1
(43) Pub. Date: Sep. 28, 2023

(54) CALCANEAL INVERSION-INDUCING ORTHOTIC SLEEVE AND METHOD OF WEARING FOR TREATING THE ANKLE

(71) Applicants: Kyle Stawitzky, Jamestown, NY (US); Christopher David Saxton, Bemus Point, NY (US)

(72) Inventors: Kyle Stawitzky, Jamestown, NY (US); Christopher David Saxton, Bemus Point, NY (US)

(21) Appl. No.: 17/701,741

(22) Filed: Mar. 23, 2022

(51) Int. Cl. A61F 5/01

(52) U.S. Cl. CPC A61F 5/0111; A61F 13/08

(57) ABSTRACT

A calcaneal inversion-inducing orthotic sleeve and method of wearing helps treat and relieve pressure off the calcaneal. The orthotic sleeve comprises a compression sock that fits over a substantial portion of the foot. The compression sock defines a toe opening, a heel opening, and an ankle opening. An ankle rim extends over the ankles for enhanced fit. The orthotic sleeve also includes a calcaneal inversion strap having a first end and a second end with hook and loop fasteners for attachment. The calcaneal inversion strap is pulled from the first end and wrapped around the base of the calcaneus (heel) from lateral to medial general. The first and second ends are attached. The calcaneal inversion strap induces a slight inversion of the ankle; whereby the orthotic reverses the anatomical abnormality from excess pronation and calcaneal eversion caused by pes planus and other etiologies, while allowing the wearer to heal and perform normal functional activities.

### FIELD OF THE INVENTION

The present invention relates generally to a calcaneal inversion-inducing orthotic sleeve and method of wearing for treating the ankle. More so, the ankle inversion-inducing orthotic sleeve provides a unique orthotic that comprises of a compression sock that fits over a substantial portion of the foot, yet has a toe opening and a heel opening; and further includes a calcaneal inversion strap that wraps around the heel, inducing a slight inversion of the ankle; whereby the orthotic reverses the anatomical abnormality from excess pronation and calcaneal eversion caused by pes planus and other etiologies, while allowing the wearer to heal and simultaneously perform normal functional activities.

### BRIEF DESCRIPTION OF THE DRAWINGS

The invention will now be described, by way of example, with reference to the accompanying drawings, in which:

FIG. 1 illustrates an anterior-lateral perspective view of the calcaneal inversion-inducing orthotic sleeve, with the calcaneal inversion strap untethered, in accordance with an embodiment of the present invention;

FIG. 2 illustrates an anterior-lateral perspective view of the calcaneal inversion-inducing orthotic sleeve, with the calcaneal inversion strap tethered, in accordance with an embodiment of the present invention;

FIG. 3 illustrates a medial perspective view of the calcaneal inversion-inducing orthotic sleeve, with the calcaneal inversion strap untethered, in accordance with an embodiment of the present invention;

FIG. 4 illustrates a medial-inferior perspective view of the calcaneal inversion-inducing orthotic sleeve, with the calcaneal inversion strap tethered, in accordance with an embodiment of the present invention; and

FIG. 5 illustrates an anterior perspective view of the calcaneal inversion-inducing orthotic sleeve, with the calcaneal inversion strap untethered, in accordance with an embodiment of the present invention;

FIG. 6 illustrates a flowchart of an exemplary method for wearing an calcaneal inversion-inducing orthotic sleeve, in accordance with an embodiment of the present invention.

Like reference numerals refer to like parts throughout the various views of the drawings.

### DETAILED DESCRIPTION OF THE INVENTION

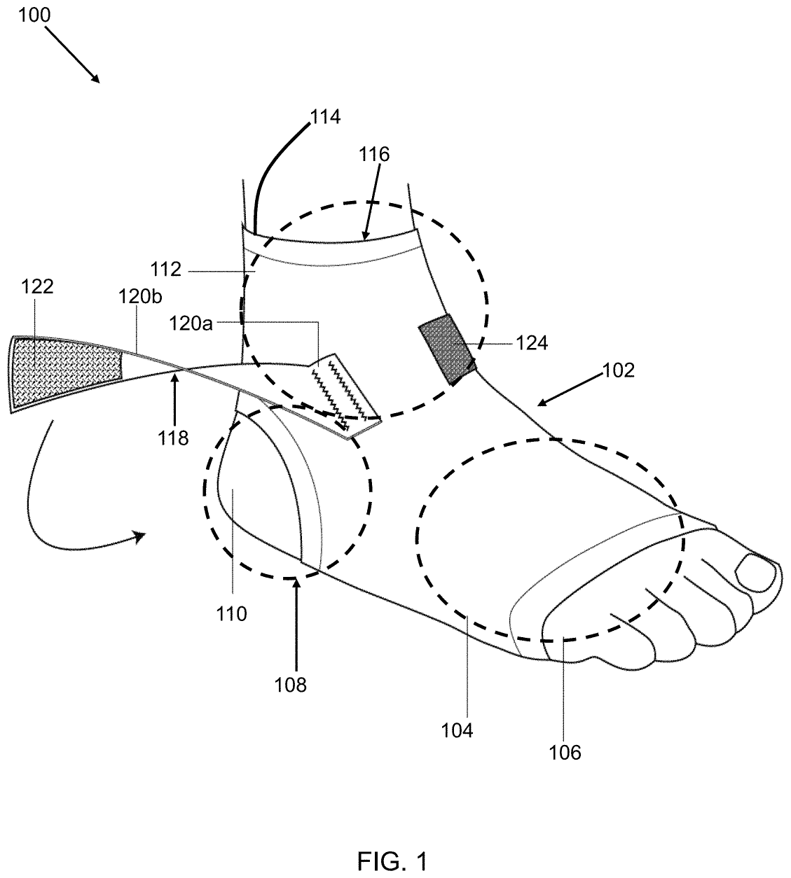

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper,” “lower,” “left,” “rear,” “right,” “front,” “vertical,” “horizontal,” and derivatives thereof shall relate to the invention as oriented in FIG. 1. Furthermore, there is no intention to be bound by any expressed or implied theory presented in the preceding technical field, background, and brief summary of the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

At the outset, it should be clearly understood that like reference numerals are intended to identify the same structural elements, portions, or surfaces consistently throughout the several drawing figures, as may be further described or explained by the entire written specification of which this detailed description is an integral part. The drawings are intended to be read together with the specification and are to be construed as a portion of the entire “written description” of this invention as required by 35 U.S.C. § 112.

In one embodiment of the present invention presented in FIGS. 1-6, an ankle inversion-inducing orthotic sleeve 100 and method 600 of wearing is configured to treat and relieve pressure off the calcaneal, and general area thereof. The calcaneal inversion-inducing orthotic sleeve 100, hereafter “orthotic sleeve 100”, provides a unique orthotic that comprises of a compression sock 102 that fits over a substantial portion of the foot, yet defines a toe opening 106 and a heel opening 110; and further includes a calcaneal inversion strap 118 that wraps around the base of the calcaneus (heel) from lateral to medial general. The calcaneal inversion strap 118 serves to induce a slight inversion of the ankle; whereby the orthotic reverses the anatomical abnormality from excess pronation and calcaneal eversion caused by pes planus and other etiologies, while allowing the wearer to heal and simultaneously perform normal functional activities

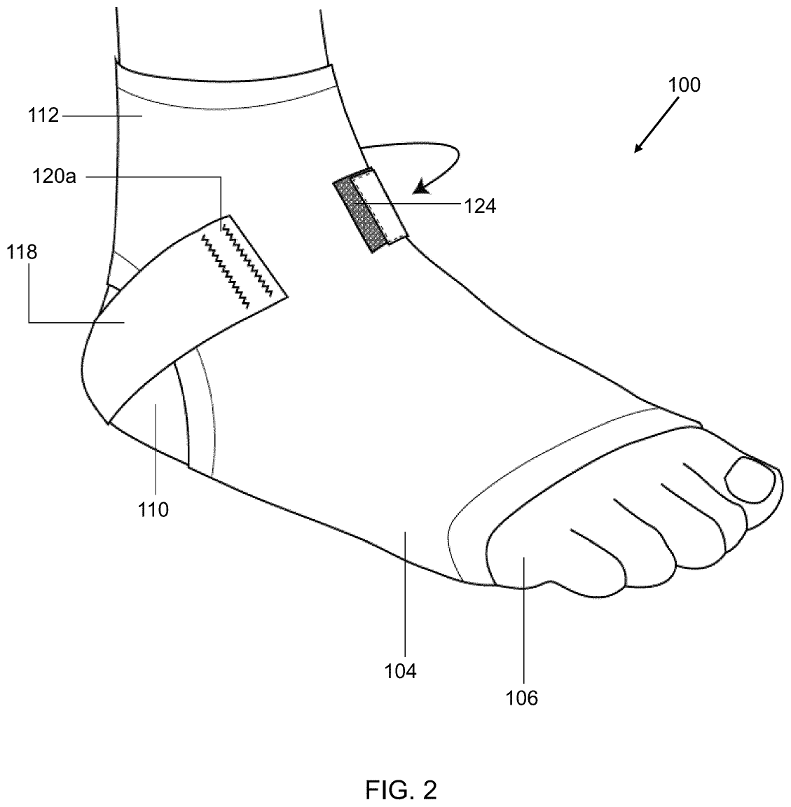

As referenced in FIG. 1, orthotic sleeve 100, when worn properly, provides simple, but effective, relief of pressure from the tarsal tunnel, posterior tibialis tendon and plantar fascia. The orthotic sleeve 100 utilizes a compression sock 102 defining a toe opening 106 and a heel opening 110, and a resilient calcaneus inversion strap 118 that attaches at a first end 120a at the anterior/superior aspect of the ankle joint, and wraps around the calcaneus lateral to medial to pull the calcaneus into inversion. Calcaneus inversion strap 118 helps relieve pressure on the posterior tibial nerve underneath the flexor retinaculum, enabling the wearer of the orthotic sleeve 100 to perform their activities of daily living without repetitive trauma and pressure to the nerve. For example, FIG. 2 illustrates an anterior-lateral perspective view of the ankle inversion-inducing orthotic sleeve, with the calcaneal inversion strap tethered.

Currently, there is no other existing orthotic that has been discovered that does what the calcaneal inversion strap 118 is intended to do. There are a myriad of ankle braces and supports that stabilize the foot and ankle. However, none of the existing orthotics focus on the inversion of the calcaneus for improved biomechanics and reduction of stress on the tarsal tunnel or origin of the plantar fascia.

In one possible embodiment, orthotic sleeve 100 is worn in conjunction with an arch support to relieve the pressure on the arch of the foot. It is known in the art that taking the pressure off of the posterior tibial nerve enables the wearer to reduce inflammation, and allows healing to occur without the repetitive provocation of the nerve that caused tarsal tunnel syndrome initially.

Orthotic sleeve 100 provides a cheap and effective alternative to the aforementioned treatment options. The orthotic is a more functional option than immobilization, a safer option than repeated injections, a cheaper option than current orthotics on the market, and also replicates the goal of physical therapy taking pressure off of the posterior tibial nerve.

In one aspect of the present invention, shown in FIG. 1, orthotic sleeve 100, comprises an elastic compression sock 102. Compression sock 102 is shaped and dimensioned to enable wearing over the foot while providing a compressive tension thereto. In one non-limiting embodiment, compression sock 102 comprises a high-elastic compression material.

Compression sock 102 comprises a toe portion 104 that is oriented towards the toes. Toe portion 104 defines a toe opening 106 that enables passage of at least a portion of the toes. Toe opening 106 is configured to provide circulation of toes. Toe opening 106 also enables the toes to function normally when ambulating.

Compression sock 102 further comprises a heel portion 108 that orients towards the heel. Heel portion 108 defines a heel opening 110 that enables passage of at least a portion of the heel. The heel opening 110 enables proper fitting and pull of inversion strap 118 around the calcaneus.

Compression sock 102 further comprises an ankle portion 112 that orients towards the ankle. Ankle portion 112 has an ankle rim 114 that at least partially extends above the ankle, when worn on the foot. This extended position of ankle rim 114 provides additional stability for the ankle. Ankle portion 112 forms an ankle opening 116 that orients towards the ankle, and that enables passage of at least a portion of the ankle.

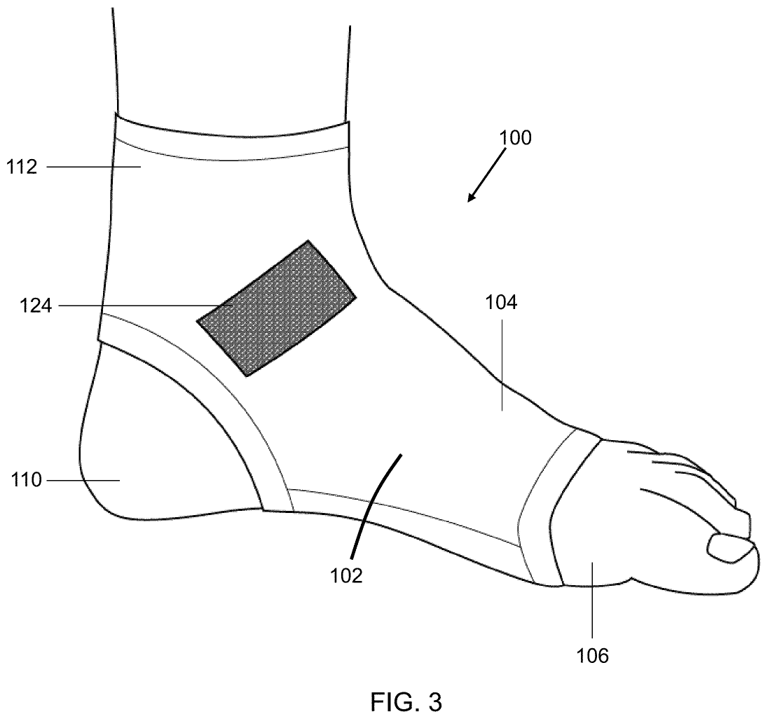

Orthotic sleeve 100 also comprises a resilient calcaneal inversion strap 118 operable with the compression sock 102. Calcaneal inversion strap 118 wraps around the base of the heel to generate an inversion tension that takes pressure off of the tarsal tunnel, posterior tibialis tendon and plantar fascia origin at the base of the calcaneus (heel). FIG. 3 illustrates a medial perspective view of the calcaneal inversion-inducing orthotic sleeve, showing calcaneal inversion strap 118 in operation.

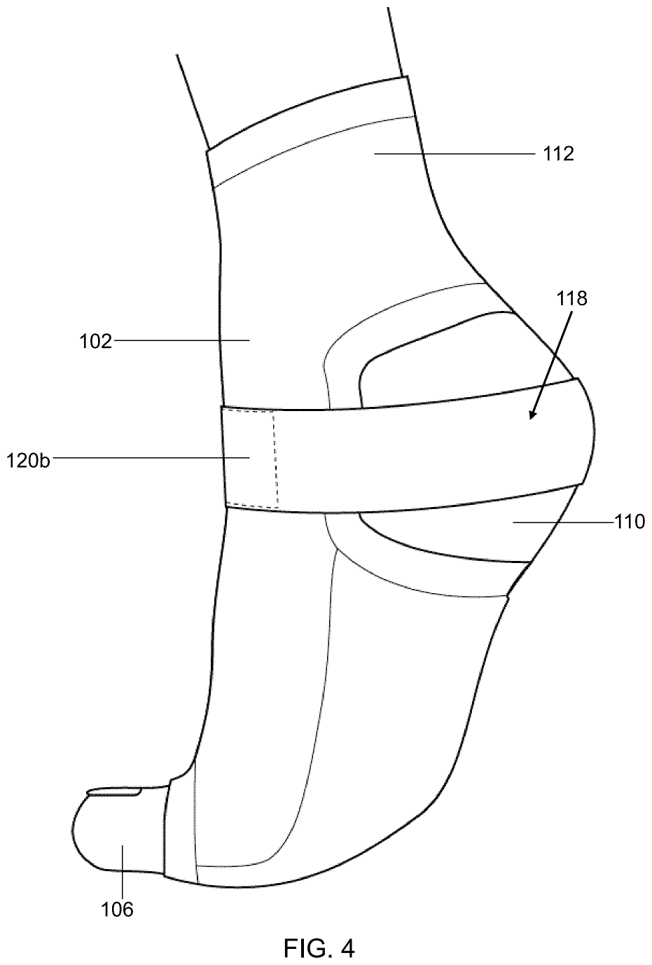

As shown in FIG. 4, calcaneal inversion strap 118 defines a first end 120a that is fixedly joined to a first anterior point on the ankle portion of the compression sock. This fixed attachment may be a stitch, an adhesive, or a mechanical fastening means known in the art. The origin of first end 120a at the anterior-superior lateral malleolus provides a good base for proper inversion when wrapping the calcaneal inversion strap 118 around the calcaneus. The calcaneal inversion strap 118 also has a second end 120b that detachably attaches to a second anterior point on the ankle region. In some embodiments, first end 120a fixedly joins the first anterior point approximately above the anterior superior-lateral aspect of the ankle joint (lateral malleolus).

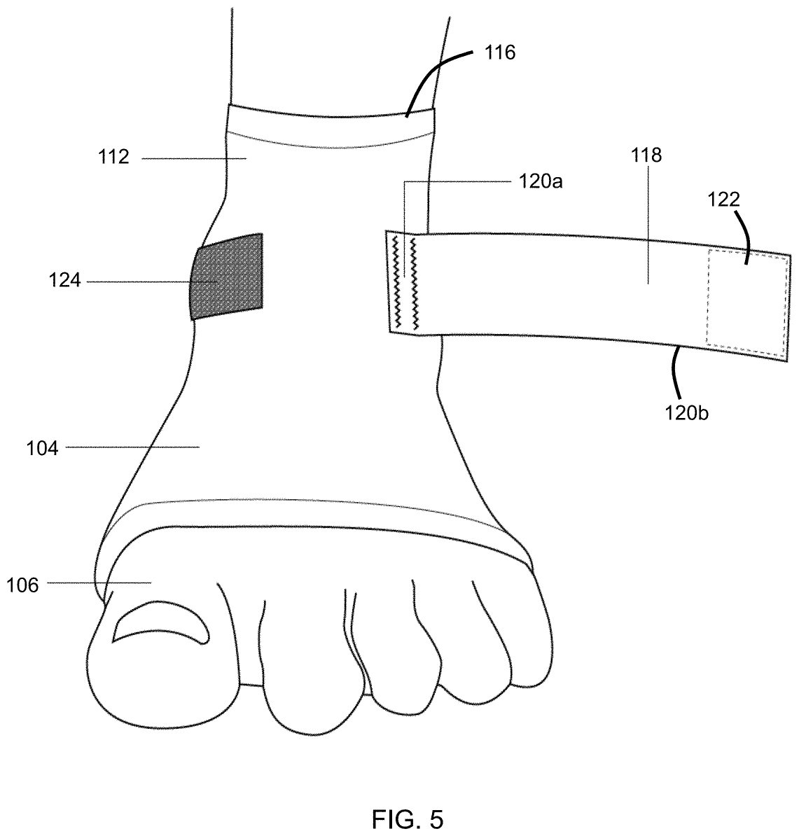

In one possible embodiment, shown in FIG. 5, first end 120a of calcaneal inversion strap 118 comprises a hook and loop fastener 122. In one embodiment, the hook and loop fastener 122 is a square patch that is sewn onto the terminus of the first end 120a. In another embodiment, the second anterior point on the ankle region of the compression sock 102 comprises a hook and loop fastener 124 configured to detachably mate with hook and loop fastener 122 on first end 120a of calcaneal inversion strap 118. However, in alternative embodiments, other fastening means may be used, including a button, an adhesive, a latch, a pin, and a hook and clasp fastening mechanism.

In operation, with the compression sock 102 being worn on the foot, the calcaneal inversion strap 118 is configured to wrap around the heel base of the heel, from lateral to medial general. In one possible embodiment, calcaneal inversion strap 118 comprises a 2″ wide adjustable strap. The tension from calcaneal inversion strap 118 pulls the heel into inversion. For example, FIG. 4 illustrates a medial-inferior perspective view of the calcaneal inversion-inducing orthotic sleeve, with the calcaneal inversion strap 118 wrapped around the base of the heel and tethered. It is known in the art that calcaneal (heel) inversion stabilizes the foot when performing functional lifts such as squats and deadlifts. The inversion of the calcaneus disperses weight throughout the foot more evenly by preventing excess pronation. This improves the biomechanics of the closed kinetic chain by promoting external rotation of the tibia and preventing valgus of the knees.

In some embodiments, compression sock 102 and calcaneal inversion strap 118 are washable. In other embodiments, the hook and loop fasteners are resistant to reduced adhesive properties from washing. In any case, the washability does not degrade the performance of orthotic sleeve 100.

One objective of the present invention is to treat, and relieve pressure off the calcaneal, and general area thereof. It is known in the art that tarsal tunnel syndrome, posterior tibialis tendonitis and plantar fasciitis are three different pathologies of the foot and ankle that can all be treated with inversion of the calcaneus and compression in the foot/ankle. Due to the excess pronation and calcaneal eversion caused by pes planus and other etiologies, an orthotic with a calcaneal inversion strap 118 can reverse this anatomical abnormality and allow the user to heal while still being able to perform normal functional activities. This orthotic sleeve 100 is also a viable option for stabilization and improved biomechanics during functional weight training, specifically for deadlifts and squats.

In another embodiment, the present disclosure provides ankle inversion-inducing compression device, which includes:
a) an elastic compression sock 102 without a toe portion 104 and a modified heel portion 108, configured to extend above the ankle, the heel portion 108 adapted to allow a strap to along from lateral to medial along the base of the calcaneus;
b) a 2-inch wide velcro-based adjustable strap with an origin at the anterior superior-lateral aspect of the ankle joint (lateral malleolus) with the ability to extend around the base of the calcaneus from lateral to medial; and
c) a Velcro attachment mechanism on the anterior-superior-medial aspect of the ankle joint (medial malleolus) to anchor the adjustable strap for inducing slight inversion of the ankle.

The calcaneal inversion-inducing compression device, wherein the sock is made of a high-elastic compression material.

The calcaneal inversion-inducing compression device of claim 1 wherein the strap is made of 2″ knit sewn elastic and permanently sewn into the anterior-superior-lateral aspect of the ankle joint (lateral malleolus).

A 2″ by 1″ Velcro attachment site sewn into the end of the knit elastic strap for adjustable attachment to the compression sock 102.

The calcaneal inversion-inducing compression device, wherein the strap is adjustable via the elasticity of the strap and its attachment via a Velcro mechanism on the anterior-superior-medial aspect of the ankle joint (medial malleolus).

The ankle inversion-inducing compression device, wherein the sock and strap are washable without damage to the Velcro or its adhesive properties

The calcaneal inversion strap 118 with compression sock 102 orthotic has been invented on the predication that inversion of the calcaneus will help multiple pathologies of the foot and ankle heal while enabling the user to ambulate without limitation due to pain or neurological symptoms. The orthotic provides compression via the pre-fabricated sock, which will help reduce edema in the foot and ankle. It is known in the art that edema is very common in lower extremity injuries due to the effect of gravity when in an upright or seated position. Furthermore, compression will give good stability to the ankle and arch.

Further, the calcaneal inversion strap 118 itself, provides an adjustable pull on the calcaneus from the anterior-superior lateral malleolus to the anterior-superior medial malleolus. The inversion of the calcaneus will take pressure off of the tarsal tunnel (including posterior tibialis nerve), posterior tibialis tendon, and origin of the plantar fascia at the calcaneus. With all of these aforementioned pathologies, pronation of the foot is a common cause of aggravation and injury. With pronation of the foot, the calcaneus everts, putting more pressure on these structures.

In an efficacious manner, the calcaneal inversion strap 118 reverses this eversion and provides support to these structures. Another common function of this orthotic is the support it provides during heavier functional lifts such as barbell squats and deadlifts. In these functional lifts, a common source of injury is the pronation of bilateral feet when performing heavy lifts. This pronation puts more force through the knees causing them to valgus. The calcaneal inversion strap 118 provides a more stable base of support in the closed kinetic chain, which will cause the tibias to externally rotate and keep the knees from valgusing.

The calcaneal inversion strap 118 with compression sock 102 orthotic would be effectively manufactured by a factory that manufactures other orthotics. The material needed should be readily available at these factories. Also, after seeing the prototype, it should be easily mass produced. The calcaneal inversion strap 118 orthotic would need to be produced in two different sizes (small/medium, large/ex-large) and for both left and right feet.

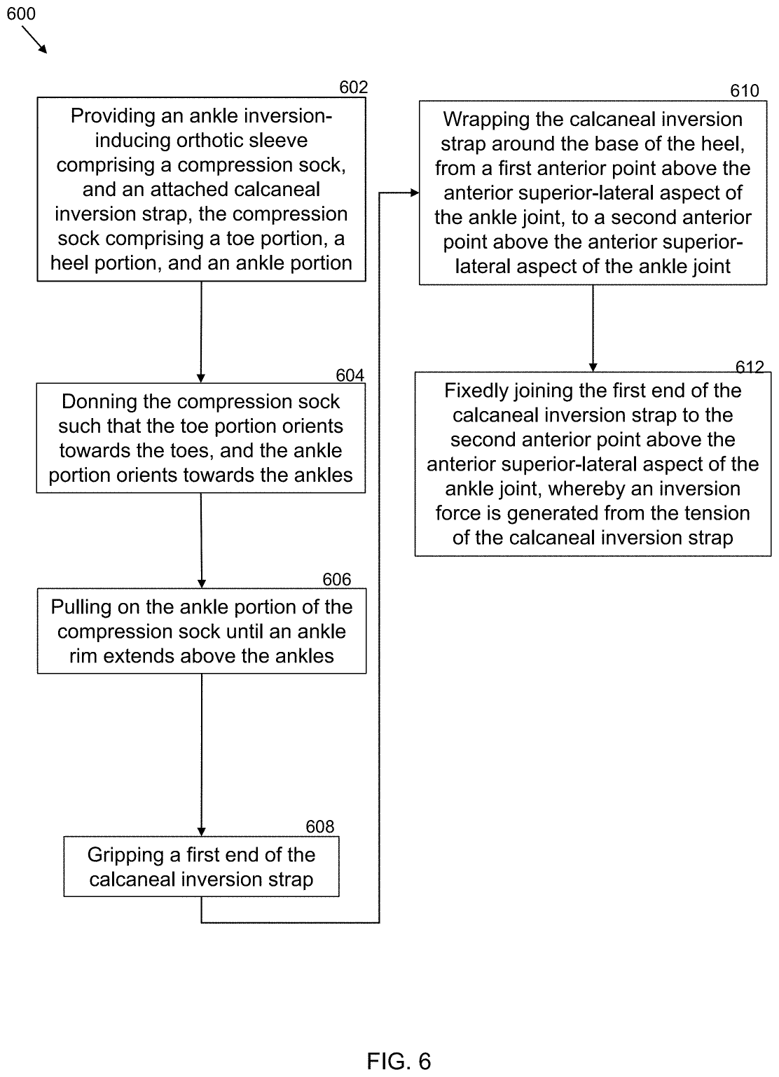

FIG. 6 illustrates a flowchart of an exemplary method 600 for wearing an calcaneal inversion-inducing orthotic sleeve. The method comprises an initial Step 602 of providing an calcaneal inversion-inducing orthotic sleeve comprising a compression sock, and an attached calcaneal inversion strap, the compression sock comprising a toe portion, a heel portion, and an ankle portion. The method 600 also includes a Step 604 of donning the compression sock such that the toe portion orients towards the toes, and the ankle portion orients towards the ankles.

Another Step 606 comprises pulling on the ankle portion of the compression sock until an ankle rim extends above the ankles. Another Step 608 may include gripping a first end of the calcaneal inversion strap. Yet another Step 610 may include wrapping the calcaneal inversion strap around the base of the heel, from a first anterior point above the anterior superior-lateral aspect of the ankle joint, to a second anterior point above the anterior superior-lateral aspect of the ankle joint. A final Step 612 comprises fixedly joining the first end of the calcaneal inversion strap to the second anterior point above the anterior superior-lateral aspect of the ankle joint, whereby an inversion force is generated from the tension of the calcaneal inversion strap.

Since many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalence.

What I claim is:

1. A calcaneal inversion-inducing orthotic sleeve, the orthotic sleeve comprising:
an elastic compression sock operable to enable wearing over the foot,
the compression sock comprising a toe portion oriented towards the toes, the toe portion defining a toe opening enabling passage of at least a portion of the toes,
the compression sock further comprising a heel portion oriented towards the heel, the heel portion defining a heel opening enabling passage of at least a portion of the heel,
the compression sock further comprising an ankle portion oriented towards the ankle, the ankle portion having an ankle rim at least partially extending above the ankle, the ankle portion forming an ankle opening oriented towards the ankle and enabling passage of at least a portion of the ankle; and
a resilient calcaneal inversion strap defining a first end fixedly joined to a first anterior point on the ankle region, and a second end detachably attachable to a second anterior point on the ankle region, the calcaneal inversion strap operable to wrap around the heel base of the heel, from lateral to medial general,
whereby the tension from the calcaneal inversion strap pulls the heel into inversion.

2. The orthotic sleeve of claim 1, wherein the calcaneal inversion strap comprises a 2-inch-wide adjustable strap.

3. The orthotic sleeve of claim 1, wherein the first end fixedly joins the first anterior point above the anterior superior-lateral aspect of the ankle joint (lateral malleolus).

4. The orthotic sleeve of claim 1, wherein the compression sock comprises a high-elastic compression material.

5. The orthotic sleeve of claim 1, wherein the first end of the calcaneal inversion strap comprises a hook and loop fastener.

6. The orthotic sleeve of claim 5, wherein the second anterior point on the ankle region of the compression sock comprises a hook and loop fastener configured to detachably mate with the hook and loop fastener on the first end of the calcaneal inversion strap.

7. The orthotic sleeve of claim 6, wherein the hook and loop fasteners are resistant to reduced adhesive properties from washing.

8. The orthotic sleeve of claim 1, wherein the compression sock and the calcaneal inversion strap are washable.

9. The orthotic sleeve of claim 1, further comprising an arch support configured to help relieve the pressure on the arch of the foot.

10. A calcaneal inversion-inducing compression device for receiving a foot comprising of:
an elastic compression sock without a toe portion and a modified heel portion, configured to extend above the ankle, the heel portion adapted to allow a strap to along from lateral to medial along the base of the calcaneus;
a 2-inch-wide Velcro-based adjustable strap with an origin at the anterior superior-lateral aspect of the ankle joint (lateral malleolus) with the ability to extend around the base of the calcaneus from lateral to medial; and
a Velcro attachment mechanism on the anterior-superior-medial aspect of the ankle joint (medial malleolus) to anchor the adjustable strap for inducing slight inversion of the ankle.

11. The calcaneal inversion-inducing compression device of claim 10, wherein the sock is made of a high-elastic compression material.

12. The calcaneal inversion-inducing compression device of claim 10, wherein the strap is made of 2″ knit sewn elastic and permanently sewn into the anterior-superior-lateral aspect of the ankle joint (lateral malleolus).

13. The calcaneal inversion-inducing compression device of claim 10, further comprising a 2 inch by 1 inch Velcro attachment site sewn into the end of the knit elastic strap for adjustable attachment to the compression sock.

14. The calcaneal inversion-inducing compression device of claim 10, wherein the strap is adjustable via the elasticity of the strap and its attachment via a Velcro mechanism on the anterior-superior-medial aspect of the ankle joint (medial malleolus).

15. The calcaneal inversion-inducing compression device of claim 10, wherein the sock and strap are washable without damage to the Velcro or its adhesive properties.

16. A method of wearing a calcaneal inversion-inducing orthotic sleeve, the method comprising:
providing an calcaneal inversion-inducing orthotic sleeve comprising a compression sock, and an attached calcaneal inversion strap, the compression sock comprising a toe portion, a heel portion, and an ankle portion;
donning the compression sock such that the toe portion orients towards the toes, and the ankle portion orients towards the ankles;
pulling on the ankle portion of the compression sock until an ankle rim extends above the ankles;
gripping a first end of the calcaneal inversion strap;
wrapping the calcaneal inversion strap around the base of the heel, from a first anterior point above the anterior superior-lateral aspect of the ankle joint, to a second anterior point above the anterior superior-lateral aspect of the ankle joint; and
fixedly joining the first end of the calcaneal inversion strap to the second anterior point above the anterior superior-lateral aspect of the ankle joint, whereby an inversion force is generated from the tension of the calcaneal inversion strap.
